(19) United States
(12) Patent Application Publication
(10) Pub. No.: US 2019/0110675 A1
(43) Pub. Date: Apr. 18, 2019

(54) APPARATUS FOR CLEANING AN IMAGING SYSTEM USED DURING A MEDICAL PROCEDURE

(71) Applicant: Titan Medical Inc., Toronto (CA)

(72) Inventors: Leonard M. Faria, Swansea, MA (US); Dustin Gaidos, Milton, MA (US); Neal H. Marshall, Ashby, MA (US); Brian A. Schopka, Worcester, MA (US); Jason P. Julian, Charlton, MA (US); Ronald F. Scharf, Sturbridge, MA (US); Randal B. Chinnock, Ashford, CT (US)

(21) Appl. No.: 16/093,918

(22) PCT Filed: Apr. 13, 2017
(86) PCT No.: PCT/CA2017/000085

§ 371 Date: Oct. 15, 2018

Related U.S. Application Data
(60) Provisional application No. 62/323,032, filed on Apr. 15, 2016.

(51) Int. Cl. A61B 1/12; A61B 1/00; A61B 1/05; A61B 1/015

(52) U.S. Cl. CPC A61B 1/126; A61B 1/00144; A61B 1/127; A61B 1/015; A61B 1/00105; A61B 1/05; A61B 1/00091

(57) ABSTRACT

A method and apparatus for cleaning at least one optical element of an imaging system is disclosed, the imaging system having a distal end inserted into a patient during a medical procedure. The apparatus includes a manifold removably receivable on the distal end of the imaging system, the manifold having an inlet for receiving a flow of cleaning fluid and including a plurality of nozzles in communication with the inlet, each nozzle being aligned to direct cleaning fluid toward the at least one optical element when the manifold is received on the distal end of the imaging system. The apparatus also includes a feed tube in communication with the inlet for supplying cleaning fluid to the manifold, the feed tube having an end for removably coupling to a cleaning fluid supply, the feed tube being routed external to the imaging system when the manifold is received on the distal end of the imaging system.

### BACKGROUND

### 1. Field

This disclosure relates generally to cleaning an optical element of an imaging system used during a medical procedure.

### 2. Description of Related Art

Imaging devices are used in many medical procedures to provide a remote view of the site of the procedure. Typical imaging systems include an endoscopic device having image capture optical elements exposed to body fluids and organs surrounding the site of the procedure. The imaging system may also include illumination optical elements for illuminating the site of the procedure. The medical procedure may involve either non-surgical imaging tasks such as diagnostic imaging or may involve a laparoscopic surgery performed through an incision in a wall of a body cavity of a patient. During the procedure, the imaging and illumination optical elements may become contaminated by blood, fat, and other tissue traces that may obscure the view of the imaging system. A wash system may be provided to clean the optical elements by directing a jet of liquid (typically saline solution) and/or a jet of gas such as carbon dioxide (CO₂), or a combination of liquid and gas.

### SUMMARY

In accordance with one disclosed aspect there is provided an apparatus for cleaning at least one optical element of an imaging system, the imaging system having a distal end inserted into a patient during a medical procedure. The apparatus includes a manifold removably receivable on the distal end of the imaging system, the manifold having an inlet for receiving a flow of cleaning fluid and including a plurality of nozzles in communication with the inlet, each nozzle being aligned to direct cleaning fluid toward the at least one optical element when the manifold is received on the distal end of the imaging system. The apparatus also includes a feed tube in communication with the inlet for supplying cleaning fluid to the manifold, the feed tube having an end for removably coupling to a cleaning fluid supply, the feed tube being routed external to the imaging system when the manifold is received on the distal end of the imaging system.

The manifold and the feed tube may be provided in a sterile packaging and coupled to a sanitized imaging system immediately prior to use during the medical procedure.

The manifold and the feed tube may be configured for a single use and are discarded following the medical procedure.

The apparatus may include a collapsible volume that sealingly receives the imaging system, the collapsible volume being operably configured to sealingly couple to an access port inserted in a wall of a body cavity of the patient placing the collapsible volume in fluid communication with the body cavity and providing access to the body cavity of the patient for a medical procedure involving surgical operations, the manifold and feed tube being enclosed within the collapsible volume.

The collapsible volume, the manifold and the feed tube may be provided in a sterile packaging and are coupled to a sanitized imaging system immediately prior to use.

The collapsible volume, the manifold and the feed tube may be configured for a single use and are discarded following the procedure.

The distal end of the imaging system may have a generally rounded cross-sectional profile and the manifold may have a generally corresponding shape that encloses at least one peripheral edge of the distal end of the imaging system when received on the distal end of the imaging system.

The manifold may be operably configured to clip onto the distal end of the imaging system.

The plurality of nozzles may be spaced apart on the manifold, each nozzle being configured to generate a fluid flow pattern that is directed toward a portion of the at least one optical element.

Nozzles in the plurality of nozzles may be angled with respect to each other to cause fluid flow produced by respective nozzles to converge toward the at least one optical element.

The at least one optical element may include at least one of a final imaging lens of an image capture portion of the image system, a window covering the final imaging lens of the image capture portion, and a window through which illumination is directed for illuminating a field of view of the image capture portion.

The at least one optical element may include an outermost optical element of a plurality of optical elements associated with illumination and capture of images by the imaging system.

The feed tube may include a first length of tube having a single bore in communication with the inlet of the manifold, and a bifurcation of the single bore of the feed tube into two separate bores for coupling to two separate fluid supplies, the two separate bores being operable to receive respective cleaning fluids through the separate bores, the single bore being operable to cause the separate cleaning fluids to be substantially blended prior to reaching the inlet of the manifold.

The separate cleaning fluids may include at least one liquid cleaning fluid and at least one gaseous cleaning fluid.

The apparatus may include a liquid supply for supplying the liquid cleaning fluid and a gas supply for supplying the gaseous cleaning fluid, the liquid supply and the gas supply being controllable to permit respective liquid and gaseous cleaning fluids to be selectively supplied through the separate bores of the feed tube.

The cleaning fluid may include at least one of a water based cleaning fluid and a gaseous cleaning fluid.

The apparatus may include a heat source in thermal communication with at least a portion of the feed tube, the heat source being operable to pre-heat the cleaning fluid to prevent fogging of the at least one optical element due to condensation.

The heat source may include an electronic circuit of the imaging system and the feed tube may include a portion routed in adjacent relationship to the imaging system to provide for thermal communication between the electronic circuit and the feed tube.

In accordance with another disclosed aspect there is provided a method for cleaning at least one optical element of an imaging system having a distal end inserted into a patient during a medical procedure. The method involves receiving a manifold on the distal end of the imaging system, the manifold having an inlet for receiving a flow of cleaning fluid and including a plurality of nozzles in communication with the inlet, each nozzle being aligned to direct cleaning fluid toward the at least one optical element. The method also involves coupling a feed tube in communication with the inlet of the manifold to a cleaning fluid supply for supplying cleaning fluid to the manifold, the feed tube being routed external to the imaging system. The method further involves controlling the cleaning fluid supply to cause cleaning fluid to be selectively supplied to the feed tube for cleaning the at least one optical element.

The method may involve sealingly receiving the imaging system within a collapsible volume, the and collapsible volume enclosing the manifold and feed tube within the collapsible volume, and sealingly coupling the collapsible volume to an access port inserted in a wall of a body cavity of the patient placing the collapsible volume in fluid communication with the body cavity and providing access to the body cavity of the patient for a medical procedure involving surgical operations.

Supplying cleaning fluid to the manifold may involve supplying at least two separate cleaning fluids to the feed tube through a bifurcated bore of the feed tube, the bifurcated bore extending partway along the feed tube and terminating in a single bore operable to cause the at least two separate cleaning fluids to blend within the single bore prior to reaching the inlet of the manifold.

The at least two separate cleaning fluids may include at least one liquid cleaning fluid and at least one gaseous cleaning fluid.

Controlling the cleaning fluid supply may involve controlling a liquid supply and a gas supply to supply cleaning fluid in sequenced order to effect the cleaning of the at least one optical element.

Controlling the cleaning fluid supply may involve supplying only liquid cleaning solution for a first period of time, supplying both liquid and gaseous cleaning fluid for a second period of time, and supplying only gaseous cleaning fluid for a third period of time.

The first, second and third periods of time may each range between 0.2 seconds and 1 second.

The method may involve pre-heating the cleaning fluid to prevent fogging of the optical element due to condensation.

Other aspects and features will become apparent to those ordinarily skilled in the art upon review of the following description of specific disclosed embodiments in conjunction with the accompanying figures.

### BRIEF DESCRIPTION OF THE DRAWINGS

In drawings which illustrate disclosed embodiments,

FIG. 1 is a perspective view of an imaging system having a cleaning apparatus attached for cleaning a window of the imaging system;

FIG. 2 is a perspective view of the imaging system shown in FIG. 1 with the cleaning apparatus detached;

FIG. 3 is a front perspective view of the cleaning apparatus shown in FIG. 1 and FIG. 2;

FIG. 4 is a rear perspective view of the a manifold of the cleaning apparatus shown in FIG. 1 and FIG. 2;

FIG. 5 is a cross sectional view of the manifold taken along a line 5-5 in FIG. 3;

FIG. 6 is a cross sectional view of an alternative manifold embodiment;

FIG. 7 is a perspective view of a collapsible sleeve enclosing the cleaning apparatus shown in FIG. 3; and

FIG. 8 is a perspective view of an imaging system embodiment showing the collapsible sleeve in use.

### DETAILED DESCRIPTION

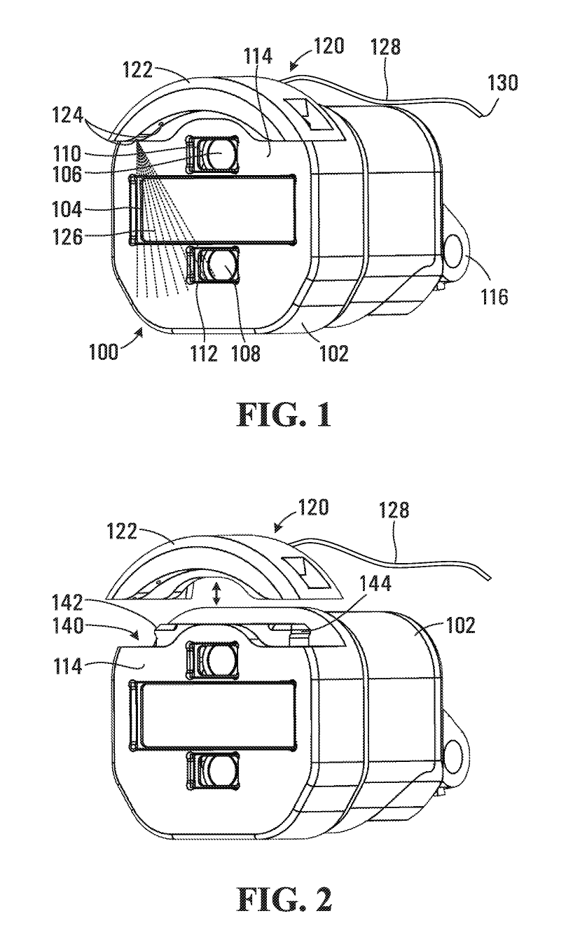

Referring to FIG. 1, an imaging system for use in a medical procedure is generally shown at 100. The imaging system 100 includes a housing 102 enclosing imaging and illumination optical elements. The imaging optical elements are covered by an optical grade window 104 that acts as an outermost imaging optical element of the imaging system 100. Illumination optical elements 106 and 108 are provided and covered by respective optical grade windows 110 and 112. The windows 104, 110 and 112 are disposed on a distal end 114 of the housing 102 and are sealed to prevent egress of body fluids into the housing 102. In other embodiments the windows 104, 110 and 112 may be omitted and outermost optical elements may be sealed to the housing 102 to act as windows for the imaging and illumination portions of the imaging system 100. Alternatively, a single window may be provided to cover the imaging optical elements and illumination optical elements 106 and 108.

The imaging portion of the imaging system 100 generally includes a plurality of lenses and one or more image sensors (not shown) disposed within the housing behind the window 104 for forming and capturing images through the window. In this embodiment the housing 102 includes a coupling 116 for coupling the imaging system 100 to shaft or manipulator (not shown in FIG. 1) suitable for maneuvering the camera to view the site of the medical procedure.

Still referring to FIG. 1, a cleaning apparatus for cleaning the windows 104, 110 and 112 of the imaging system 100 is shown generally at 120. The cleaning apparatus 120 includes a manifold 122 received on the distal end 114 of the imaging system 100. The manifold 122 includes a plurality of nozzles 124 (of which two nozzles 124 are visible in FIG. 1). The nozzles 124 are aligned to direct cleaning fluid 126 toward the imaging window 104 and the illumination windows 110 and 112. The manifold 122 also includes an inlet at the rear (not shown in FIG. 1) in communication with the plurality of nozzles 124.

The cleaning apparatus 120 also includes a feed tube 128 in communication with the inlet of the manifold 122 for supplying cleaning fluid to the manifold. The feed tube 128 has an end 130 for removably coupling to a cleaning fluid supply (not shown). In operation, a cleaning fluid 126, such as liquid saline water and/or carbon dioxide (CO₂) is supplied via the feed tube 128 to the inlet of the manifold 122, and is distributed to the plurality of nozzles 124 to cause cleaning fluid to be directed over the windows 104, 110 and 112 for removing accumulated contaminants. In the embodiments shown, the feed tube 128 is routed external to the imaging system 100 when the manifold is received on the distal end 114 of the imaging system.

Since the cleaning fluid 126 is a foreign substance introduced into the site of the medical procedure, it is an advantage to minimize an amount of cleaning fluid used. Accordingly the nozzles 124, inlet, and feed tube 128 may have a relatively narrow bore and operate at relatively high pressure to achieve sufficient cleaning action while minimizing fluid delivery. In one embodiment the diameter of the nozzles 124 is about 0.3 mm and the fluid line has an inside diameter of about 0.8 mm.

Referring to FIG. 2, the manifold 122 is configured such that the cleaning apparatus 120 is removably attached to the distal end 114 of the imaging system 100. In this embodiment the distal end 114 has a generally rounded cross-sectional profile and the manifold 122 has a generally corresponding shape that mounts on a peripheral edge 140 the imaging system 100. Additionally, the housing 102 includes a pair of clips 142 and 144 that engage corresponding features on the manifold 122 for securing the manifold to the peripheral edge 140. In one embodiment the manifold 122 is formed from a thermoplastic material that allows the manifold to deform to provide the clip fastening feature of the manifold. The feed tube 128 in this embodiment remains attached to the manifold 122 and forms part of the cleaning apparatus. The manifold 122 and feed tube 128 of the cleaning apparatus 120 may be supplied in a sterile packaging as a limited or single use component that is discarded following use in a medical procedure. Limited use of the cleaning apparatus 120 avoids the need for cleaning and sterilizing, which may easily block due to the narrow bore of the nozzles 124.

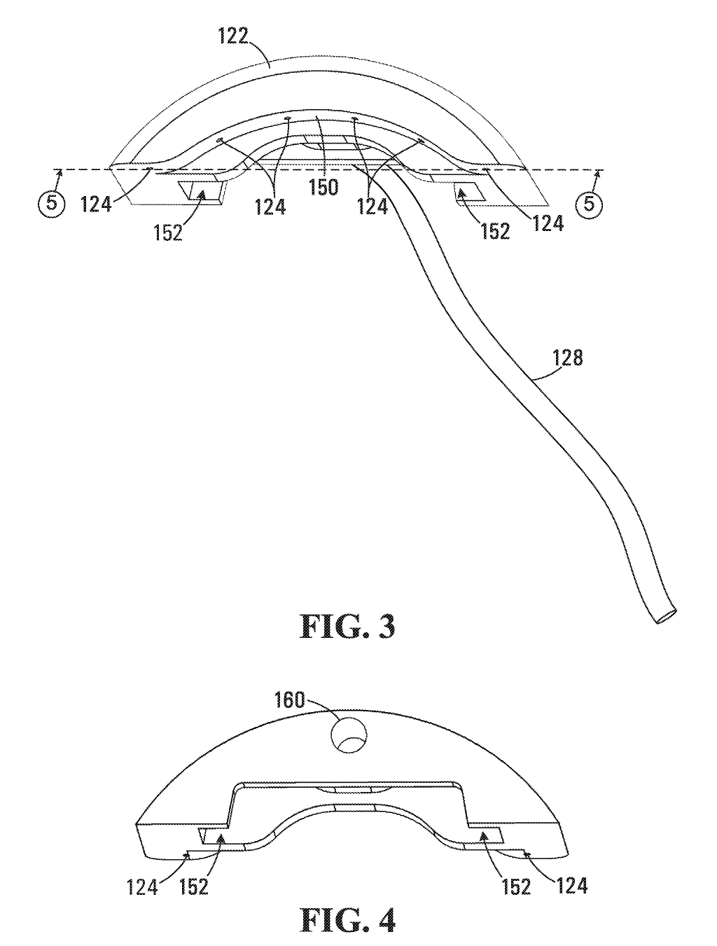

An underside of the manifold 122 is shown in perspective view in FIG. 3. Referring to FIG. 3, the manifold 122 includes six nozzles 124 spaced apart to cause each nozzle 124 to generate a fluid flow pattern that is directed toward a portion of the windows 104, 110 and 112 to provide adequate cleaning coverage. In this embodiment the manifold 122 includes an arched section 150 that also causes the nozzles to be angled with respect to each other to cause a converging fluid flow toward the windows 104, 110 and 112 that cover the imaging and illumination optical elements. In other embodiments where a single window replaces the windows 104, 110 and 112, the arched section 150 would cause the nozzles to cause a converging fluid flow toward areas of the window covering the imaging and illumination optical element. The manifold 122 also includes an aperture 152 that is received on the pair of clips 142 and 144 for locating and securing the manifold 122 on the distal end 114 of the imaging system 100. A rear side of the manifold 122 is shown in FIG. 4, revealing the inlet 160 which receives the feed tube 128 (not shown in FIG. 4). In one embodiment the feed tube 128 is received in the inlet 160 and an adhesive is introduced to retain the feed tube in the inlet. In other embodiments the inlet 160 may be sized to provide a friction fit of the feed tube 128.

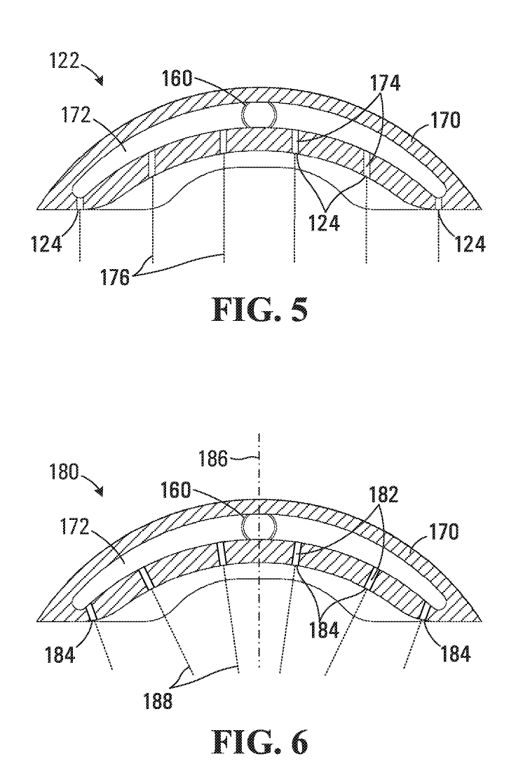

The manifold 122 is shown in cross section in FIG. 5, the cross section being taken along the line 5-5 in FIG. 3. Referring to FIG. 5, the manifold 122 includes a body 170 having a channel 172 formed within the body that has an arched shape and extends across the manifold. The channel 172 is in communication with the inlet 160 at the rear of the body 170. A plurality of conduits 174 are also formed within the body 170 in communication with the channel 172, each conduit terminating to form one of the plurality of nozzles 124. The channel 172 receives a flow of cleaning fluid from the inlet 160 and distributes the cleaning fluid across the conduits 174 to provide a flow of cleaning fluid through each nozzle 124. In this embodiment the flow of cleaning fluid is directed downwardly with respect to the manifold 122 as shown by the broken lines 176. In one embodiment the manifold 122 is formed from a thermoplastic material in two or more parts and the parts are glued together or ultrasonically welded to form the unitary manifold. One or more of the conduits 174 may be shaped to cause the flow of cleaning fluid to be directed in different flow patterns from narrow flow pattern to a spray-like wide flow pattern. Alternatively, only a portion where the flow of cleaning fluid exits the one or more conduits 174 may be angled or shaped to direct the fluid to flow in different flow patterns.

Referring to FIG. 6, an alternative embodiment of a manifold is shown at 180. The manifold 180 includes the same body 170, channel 172, and inlet 160 as the manifold 122 shown in FIG. 5, but has the plurality of conduits 182 angled inwardly such that the respective nozzles 124 each direct cleaning fluid jets at different angles to a centerline 186, as shown by the broken lines 188 in FIG. 6. As such, the manifold 180 is operable to direct the cleaning fluid flow toward a window or other optical element located proximate the centerline 186.

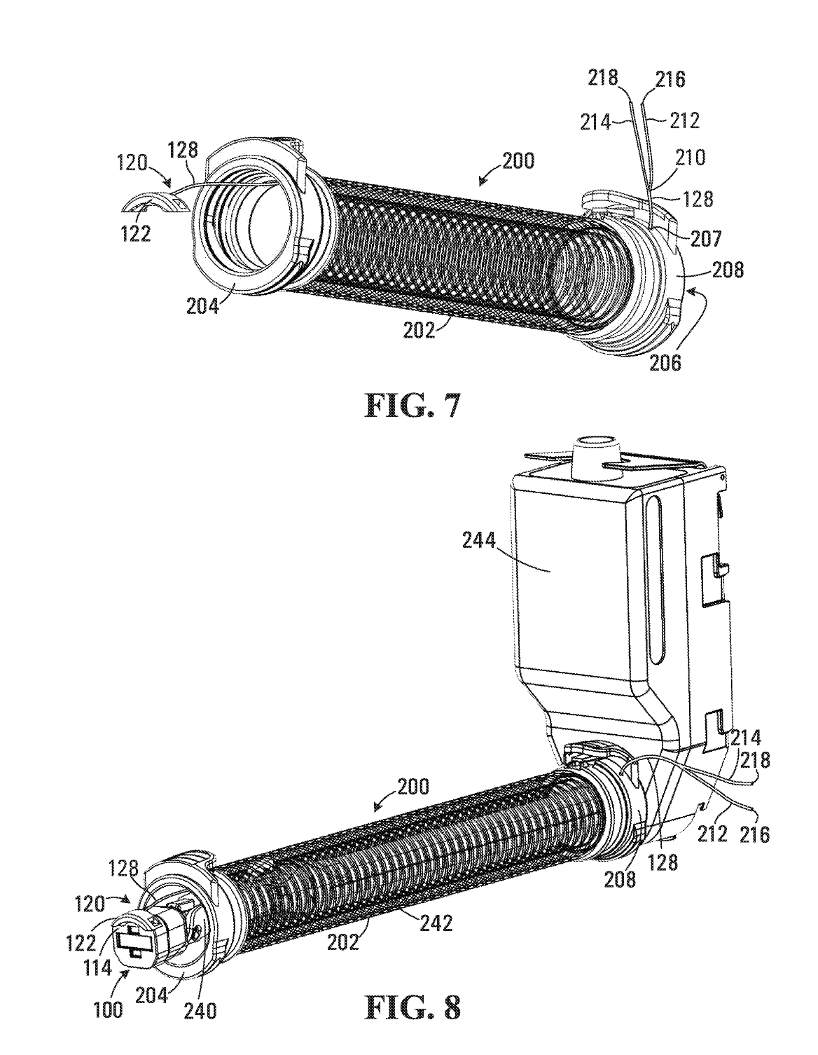

Referring to FIG. 7, in one embodiment the cleaning apparatus 120 may be included as part of a collapsible sleeve 200 that encloses the imaging system 100 when in use to perform a medical procedure. The collapsible sleeve 200 is described in commonly owned PCT Patent Application No.: PCT/CA2016/000054 filed on Feb. 26, 2016 and entitled “METHOD AND APPARATUS FOR PROVIDING ACCESS FOR A SURGICAL PROCEDURE”, which is incorporated herein by reference in its entirety. The collapsible sleeve 200 includes a collapsible volume 202 having an access coupler 204 for coupling to an access port (not shown) inserted in an incision in a body cavity wall of the patient providing access to the body cavity of the patient for a surgical procedure. The collapsible sleeve 200 also has an opening 206 for sealingly receiving the imaging system 100. The feed tube 128 is routed through the collapsible volume 202 and emerges at a sealed feed-through 207 at a proximate coupler 208. In this embodiment the feed tube 128 includes a single bore running along a first length of the tube between the manifold 122 and the point at which the tube emerges from the proximate coupler 208. The feed tube 128 also includes a bifurcation 210 of the single bore of the feed tube into two separate bores associated with lines 212 and 214 for coupling to two separate fluid supplies. In this embodiment the lines 212 and 214 have respective open ends 216 and 218 but in other embodiments the ends may terminate in a couple for coupling to a cleaning fluid supply.

An embodiment showing the collapsible sleeve 200 in use is depicted in FIG. 8. Referring to FIG. 8, in this embodiment the imaging system 100 includes a deployment boom 240 that facilitates movement of the imaging system for viewing the site of the medical procedure. The deployment boom 240 is mounted on a shaft 242 (shown within the collapsible volume 202). The shaft 242 extends outwardly from a housing 244 that encloses various drive systems for actuating movement of the deployment boom 240 and shaft 242.

The collapsible sleeve 200 and cleaning apparatus 120 may be initially provided as a unitary assembly in sterile packaging. The imaging system 100, deployment boom 240, shaft 242, and housing 244 may be separately sterilized and then inserted through the opening 206 (shown in FIG. 7) into the collapsible volume 202 and the proximate coupler 208 coupled to the housing 244 to provide a sealed volume enclosing the shaft 242. The open ends 216 and 218 of the lines 212 and 214 are then coupled to respective cleaning fluid supplies such as a saline solution and a CO₂ supply. In other embodiments the housing 244 may include connections for the line ends 216 and 218 and supply of cleaning fluids to the lines 212 and 214 may be through the housing 244. The manifold 122 is then clipped onto the periphery of the distal end 114 of the imaging system 100. The feed tube 128 is thus routed external to the imaging system 100 and along the outside of the deployment boom 240 and shaft 242. This configuration has the advantage of allowing the cleaning apparatus 120 to be provided as a single use or limited use item and simplifies cleaning of the imaging system 100 since there are no narrow bore feed lines extending through the shaft 242 that could become blocked.

Once the imaging system 100 and collapsible sleeve 200 have been assembled as shown in FIG. 8, the access coupler 204 may be coupled to the access port placing the body cavity of the patient in fluid communication with the interior of the collapsible volume 202. The volume around the shaft 242 within the collapsible volume 202 thus forms part of an insufflation volume in the body cavity of the patient that is maintained to improve access for surgical operations within the body cavity.

In the embodiment shown in FIG. 8, the housing 244 is configured to couple to a central robotic surgery unit (not shown) that includes systems for supplying the cleaning fluid. For example, the central robotic unit may include a liquid saline supply and peristaltic pump controlled by the robotic unit for supplying saline solution through one of the outlets 246 and 248. The central robotic unit may also include a gaseous CO₂ supply and a valve controlled by the robotic unit for supplying CO₂ through the other of the outlets 246 and 248.

Cleaning of the windows 104, 110 and 112 may involve a sequenced cleaning of liquid-gas/liquid and gas only. For example, initial cleaning may involve a 0.5 second burst of saline solution during a first period of time, followed by a combined burst of saline and CO₂ for a second period of time (for example 0.5 seconds). The gaseous and liquid flows in the respective lines 212 and 214 combine at the bifurcation 210 and the single bore along the feed tube 128 cause the liquid and gas to be substantially blended while traveling along the tube during the second period of time. Finally, the flow of liquid saline is discontinued during a third period of time and the CO₂ causes droplets on the windows 104, 110 and 112 to be blown off completing the cleaning cycle.

The first, second, and third periods of time may be selected to suit the imaging application. For example, during a surgical procedure, it may be desirable to limit occlusion of the surgeon's view provided by the imaging system 100 to a few seconds. A time of between about 0.2 and 1 second for each of the first, second, and third periods of time would result in a wash cycle duration of between about 0.6 seconds and about 3 seconds. In other embodiments such as diagnostic or visual observation for example, the wash cycle duration may be longer potentially providing for enhanced cleaning that would benefit such a procedure. In other embodiments, other sequences may be utilized that involve the use and a defined period for each of a type of fluids such as saline and CO₂.

In other embodiments, separate feed tubes may be provided running to the manifold 122 and the blending may occur within the manifold or some of the plurality of nozzles 124 may be used to supply liquid cleaning fluid while others supply gaseous cleaning fluid.

In one embodiment the imaging system 100 and/or the deployment boom 240 may include electronic circuitry for controlling the imaging system and transmitting image data back to the central robotic unit. The circuitry will necessarily generate heat and the feed tube 128 running adjacent to the imaging system 100, deployment boom 240, and shaft 242 will be subjected to a degree of thermal communication with the electronic circuitry at least partially pre-heating the cleaning fluid. Advantageously, the pre-heating of the cleaning fluid may prevent fogging of the windows 104, 110 and 112 due to condensation. Condensation may be caused when a cleaning fluid having a lower temperature than a surface temperature of the optical window flows over the window causing cooling of the window such that moisture within the abdominal cavity condenses on window. Condensation generally results in droplet or a film forming on the window causing fogging that interferes with imaging and/or illumination.

While the cleaning apparatus 120 is shown in conjunction with a specific example of an imaging system for performing robotic surgery, the apparatus may be used to advantage with other medical imaging instruments such as endoscopes, for example. The cleaning apparatus 120 described in the various disclosed embodiments has the advantage of being separated from the imaging system 100, facilitating cleaning and or replacement without requiring disassembly of the imaging system or other components. The cleaning apparatus 120 may also be fabricated from relatively inexpensive materials and supplied in a sterile condition for single use or limited use.

While specific embodiments have been described and illustrated, such embodiments should be considered illustrative of the invention only and not limiting as construed in accordance with the accompanying claims.

1. An apparatus for cleaning at least one optical element of an imaging system, the imaging system including a distal end inserted into a patient during a medical procedure, the apparatus comprising:
a manifold removably receivable on the distal end of the imaging system, the manifold including an inlet configured to receive a flow of cleaning fluid and a plurality of nozzles in communication with the inlet, each nozzle being aligned to direct cleaning fluid toward the at least one optical element when the manifold is received on the distal end of the imaging system; and
a feed tube in communication with the inlet configured to supply cleaning fluid to the manifold, the feed tube including an end configured to be removably coupled to a cleaning fluid supply, the feed tube being routed external to the imaging system when the manifold is received on the distal end of the imaging system.

2. The apparatus of claim 1 wherein the manifold and the feed tube are provided in a sterile packaging and coupled to a sanitized imaging system immediately prior to use during the medical procedure.

3. (canceled)

4. The apparatus of claim 1 further comprising a collapsible volume that sealingly receives the imaging system, the collapsible volume configured to sealingly couple to an access port inserted in a wall of a body cavity of the patient placing the collapsible volume in fluid communication with the body cavity and providing access to the body cavity of the patient for a medical procedure involving surgical operations and wherein the manifold and feed tube are enclosed within the collapsible volume.

5. The apparatus of claim 4 wherein the collapsible volume, the manifold and the feed tube are provided in a sterile packaging and are coupled to a sanitized imaging system immediately prior to use.

6. (canceled)

7. (canceled)

8. (canceled)

9. The apparatus of claim 1 wherein the plurality of nozzles are spaced apart on the manifold, each nozzle being configured to generate a fluid flow pattern that is directed toward a portion of the at least one optical element.

10. The apparatus of claim 9 wherein nozzles in the plurality of nozzles are angled with respect to each other to cause fluid flow produced by respective nozzles to converge toward the at least one optical element.

11. The apparatus of claim 1 wherein the at least one optical element comprises at least one of:
a final imaging lens of an image capture portion of the image system;
a window covering the final imaging lens of the image capture portion; or
a window through which illumination is directed for illuminating a field of view of the image capture portion.

12. The apparatus of claim 1 wherein the at least one optical element comprises an outermost optical element of a plurality of optical elements associated with illumination and capture of images by the imaging system.

13. The apparatus of claim 12, wherein the feed tube comprises:
a first length of tube including a single bore in communication with the inlet of the manifold; and
a bifurcation of the single bore of the feed tube into two separate bores configured to be coupled to two separate fluid supplies, the two separate bores being operable to receive respective cleaning fluids through the separate bores, the single bore being operable to cause the separate cleaning fluids to be substantially blended prior to reaching the inlet of the manifold.

14. The apparatus of claim 13 wherein the separate cleaning fluids comprise at least one liquid cleaning fluid and at least one gaseous cleaning fluid.

15. The apparatus of claim 14 further comprising a liquid supply configured to supply the liquid cleaning fluid and a gas supply configured to supply the gaseous cleaning fluid, the liquid supply and the gas supply being controllable to permit respective liquid and gaseous cleaning fluids to be selectively supplied through the separate bores of the feed tube.

16. (canceled)

17. The apparatus of claim 1 further comprising a heat source in thermal communication with at least a portion of the feed tube, the heat source being operable to pre-heat the cleaning fluid to prevent fogging of the at least one optical element due to condensation.

18. The apparatus of claim 17 wherein the heat source comprises an electronic circuit of the imaging system and wherein the feed tube comprises a portion routed in adjacent relationship to the imaging system to provide for thermal communication between the electronic circuit and the feed tube.

19. A method for cleaning at least one optical element of an imaging system including a distal end inserted into a patient during a medical procedure, the method comprising:
receiving a manifold on the distal end of the imaging system, the manifold including an inlet configured to receive a flow of cleaning fluid and including a plurality of nozzles in communication with the inlet, each nozzle being aligned to direct cleaning fluid toward the at least one optical element;
coupling a feed tube in communication with the inlet of the manifold to a cleaning fluid supply configured to supply cleaning fluid to the manifold, the feed tube being routed external to the imaging system; and
controlling the cleaning fluid supply to cause cleaning fluid to be selectively supplied to the feed tube for cleaning the at least one optical element.

20. The method of claim 19 further comprising:
sealingly receiving the imaging system within a collapsible volume, the collapsible volume enclosing the manifold and feed tube within the collapsible volume; and
sealingly coupling the collapsible volume to an access port inserted in a wall of a body cavity of the patient placing the collapsible volume in fluid communication with the body cavity and providing access to the body cavity of the patient for a medical procedure involving surgical operations.

21. The method of claim 19, wherein supplying cleaning fluid to the manifold comprises supplying at least two separate cleaning fluids to the feed tube through a bifurcated bore of the feed tube, the bifurcated bore extending partway along the feed tube and terminating in a single bore operable to cause the at least two separate cleaning fluids to blend within the single bore prior to reaching the inlet of the manifold.

22. The method of claim 21 wherein the at least two separate cleaning fluids comprise at least one liquid cleaning fluid and at least one gaseous cleaning fluid.

23. The method of claim 22 wherein controlling the cleaning fluid supply comprises controlling a liquid supply and a gas supply to supply cleaning fluid in sequenced order to effect the cleaning of the at least one optical element.

24. The method of claim 23 wherein controlling the cleaning fluid supply comprises:
supplying only liquid cleaning solution for a first period of time;
supplying both liquid and gaseous cleaning fluid for a second period of time; and
supplying only gaseous cleaning fluid for a third period of time.

25. (canceled)

26. The method of claim 19 further comprising pre-heating the cleaning fluid to prevent fogging of the optical element due to condensation.
